(19) United States
(12) Patent Application Publication
(10) Pub. No.: US 2006/0189940 A1
(43) Pub. Date: Aug. 24, 2006

(54) Implant positioning system and method

(76) Inventor: Andrew Jared Kirsch, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 11/065,310

(22) Filed: Feb. 24, 2005

(51) Int. Cl. A61M 5/178; A61B 5/02

(52) U.S. Cl. 604/164.1; 600/500

(57) ABSTRACT

A system for delivering an injectable material to a site within a mammal, the system comprising a cannula and a needle. The cannula defining an aperture in a side wall thereof and comprising an angled ramp platform disposed within the cannula proximate to the aperture that extends proximally and inwardly away from proximate the distal end of the aperture. The needle is longitudinally movable within the cannula between a withdrawn position, at which a tip of the needle is stored within the cannula, to an extended position, at which the tip of the needle extends through the aperture of the cannula a predetermined distance from the side wall of the cannula. In one aspect, the system also includes a supply of injectable biocompatible bulking composition for controlling vesicoureteral reflux that is operatively connected to the needle.

### FIELD OF THE INVENTION

The invention relates to a medical system and method of using same. More particularly, the invention relates to a medical system and method for positioning and implanting of biocompatible compositions in the human body. Still more particularly, the invention relates to a medical system and method for delivering a biocompatible bulking composition to a urological site to treat vesicoureteral reflux or urinary incontinence.

### BACKGROUND OF THE INVENTION

Vesicoureteral reflux (urinary reflux or VUR) is a condition wherein urine moves from the bladder into the ureters and sometimes the kidney during voiding or with elevated pressure in the bladder. VUR is common in children with anatomic abnormalities of the urinary tract; however, it also occurs in children with anatomically normal but infected urinary tracts.

Normally, the junction of the terminal ureter with the urinary bladder provides a competent sphincter so that during micturition urine leaves the bladder only via the urethra. Reflux occurs when there is inadequate intravesical submucosal tunnel (valve mechanism) or defective attachment of the ureter to the bladder. Thus, an anatomically impaired vesicoureteral junction facilitates reflux of urine and bacteria into the ureters and can result in upper tract infection and renal damage.

VUR can develop before birth (prenatally), if the anatomy of the bladder and ureters does not form sufficiently to control the direction of flow of the urine. In these patients, the VUR is normally more severe. Alternatively, VUR can develop after birth as the child grows. Often the presence of VUR is only diagnosed once the child has suffered UTIs. In severe cases, this can mean that the kidney is already damaged to some extent on diagnosis.

As the child with VUR grows and develops, spontaneous resolution of the condition may occur over time. However, the rate of spontaneous resolution also decreases with increasing patient age. Resolution of reflux cannot be expected to occur universally and the longer the child is left without medical intervention, the more likely they are to suffer from the long-term consequences associated with VUR. This is particularly important in children under age 3 who are at the greatest risk for renal scarring after infection.

Reflux of urine into the ureter, and more severely into the kidneys, will exacerbate any UTI as it facilitates progression of the infection into the upper urinary tract. Particularly in more severe cases, reflux of infected urine increases the risk of pyelonephritis—an inflammation of the kidney and pelvis—which can have longer-term consequences. Recurrent pyelonephritic infection can lead to damage within the renal unit and tissue scarring. Further, scarring can occur congenitally in children who have developed VUR prenatally, or can be a secondary effect of persistent infections in the urinary tract during childhood. Scarring of the renal unit can have long-term serious consequences, which include reduced renal function and hypertension.

The aim of VUR treatment, therefore, is to reduce the risk of UTI, particularly febrile UTIs within the upper urinary tract and pyelonephritis, thus decreasing morbidity. Treatment may also avoid the risk of further damage to a renal unit that has already been weakened.

The initial treatment of vesicoureteral reflux usually consists of suppressive antibiotics in anticipation of spontaneous resolution. Thus, reflux of minor degree may disappear with standard treatment for intercurrent urinary infection, i.e., antibiotics. For more severe degrees of reflux and minor reflux unresponsive to antibiotics, surgical correction is usually necessary. However, open surgical repair has a well recognized morbidity, which consists of pain and immobilization of a lower abdominal incision, as well as the attendant risks of surgery in general, e.g., anesthesia.

An alternative to open surgery which reduces the morbidity of vesicoureteral reflux correction would be of significant clinical benefit. Such a technique has been developed over in the past and is termed endoscopic subureteral injection. Endoscopic subureteral injection is a simple procedure in which a substance is injected into the subureteral region, usually on an outpatient basis. The injected substance implants in the subureteral region and enhances its bulk. In turn, the enhanced bulk of the subureteral region narrows the ureteral orifice thereby inhibiting retrograde urine flow. Hence, the injected substance used as an implant material should be non-migratory, conserve its volume, be non-antigenic and be able to be delivered endoscopically. To date, endoscopic subureteral injection of various substances has had varying results. For example, polytetrafluorethylene (Teflon™) was one of the first substances utilized. However, Teflon™ has been found to induce a granulomatous reaction and is capable of migration to distant areas such as the pelvic lymph nodes, spleen, lungs and brain. These observations have cast doubt on the applicability of use in humans of Teflon™.

Another substance that has been utilized is glutaraldehyde cross-linked bovine dermal collagen. The major problem with the use of collagen in the treatment of reflux is that the implant is biodegradable and, hence, volume size of the implant decreases with time. This has made re-treatment necessary. Collagen is also quite viscous and therefore difficult to inject.

Another injectable substance that has been used for the treatment of vesicoureteral reflux is a chondrocyte-alginate suspension. Chondrocytes, cells capable of synthesizing cartilage, were harvested from animals, mixed with alginate to form a gel that was subsequently injected into the subureteral region of the animal. Although encouraging results have been seen, use of this suspension requires a biopsy in order to harvest the chondrocytes with the patient having to return at a later date to undergo treatment with the autologous chondrocyte-alginate suspension.

Additionally, displacement of the implant, either distal or lateral to the ureteral orifice, usually in a position inadequate to provide support for the submucosal tunnel, has been a problem with all of the above mentioned substances.

Urinary incontinence is an inability to hold urine in the bladder until it is decided to release the urine. Urinary incontinence is an extremely common problem. Women are nearly twice as likely to experience incontinence as men. This is likely due to pregnancy and childbirth, menopause, and the structure of the female urinary tract. However, both men and women can experience incontinence due to strokes, multiple sclerosis, prostate surgery and old age. As a result, when pressure is exerted on these tissues by coughing, lifting, etc., urine is involuntarily discharged from the bladder through the urethra.

There are many types of urinary incontinence, such as, for example, stress incontinence, urge incontinence, functional incontinence, overflow incontinence, transient incontinence, and mixed types of incontinence. Typically, these types of urinary incontinence are each related to particular physical problems, such as weakened muscles, physical changes, physiological changes, neurological problems, and disease and are generally suitable for treatment via a variety of surgical and non-surgical procedures.

Among the available non-surgical treatments are Kegel exercises to strengthen the pelvic-floor muscles, electrical stimulation of pelvic muscles, biofeedback, timed voiding, bladder training, and medications. Some choose to wear absorbent pads or undergarments. Another choice includes restricting certain liquids. However, each treatment has limited effectiveness and often potentially harmful side effects. For instance, Kegel exercises and electrical stimulation can reduce stress and urge incontinence. Biofeedback may relieve stress and urge incontinence. Medications can reduce certain types of leakage by inhibiting contractions or relaxing muscles. These treatments do not cure urinary incontinence and generally only alleviate the problem. Further, certain medications can have harmful side effects, such as the increased risk of breast and endometrial cancer associated with estrogen therapy.

If the non-surgical treatments are ineffective, open surgical repair of the bladder neck is often attempted. Many surgical procedures are also available to treat urinary incontinence. Among the available procedures are pessaries, implants, bladder surgery, and catheterization. Surgical options include surgery to pull the bladder up to a more normal position, surgery to secure the bladder with a wide sling, or surgery to insert an artificial sphincter around the urethra. However, again, each has its own effectiveness rate and possible side effects. For example, one serious concern with the use of pessaries or long-term catheters is urinary tract infections. Further, such surgical repair procedures are not successful for all patients. Moreover, there are always certain risks associated with open surgical procedures, such as infection, risks of anesthesia, etc.

As an alternative to surgical repair, urinary incontinence has been treated by periurethral injection therapy, in which a substance is injected into the tissue surrounding the urethra, i.e., the periurethral tissue, to add bulk to this tissue. The aim of this treatment is to restore the proximal urethra to its proper normally closed condition and to keep it closed during coughing, straining, or exercise. The injected substance compresses the urethra proximate the level of the bladder neck to impede the involuntary flow of urine from the bladder.

Many injectable substances have been tried for this purpose with varying results. For example, in the first half of the twentieth century sclerosing solutions, such as sodium morrhuate or cod liver oil, were injected into the anterior vaginal wall. An inflammatory response developed with secondary scarring which resulted in compression of the incompetent urethra. Although this material was successful in curing incontinence in some patients, complications included pulmonary infarction and cardiac arrest. Similarly, paraffin and other sclerosing solutions have been tried with poor results.

Polytetrafluoroethylene particles (TEFLON™, POLYTEF™) have been used as an injectable material for the correction of urinary incontinence with a success rate of from 30% to 86% in some studies. As noted above in the discussion of VUR, complications associated with this procedure included foreign body granulomas that ended to migrate to distant organs, such as the lungs, liver, spleen and brain. Resultantly, the use of polytetrafluoroethylene particles is currently disfavored.

As noted in the discussion of VUR above, another injectable material that has been used recently for the treatment of urinary incontinence is glutaraldehyde cross-linked bovine dermal collagen. A major problem associated with the use of collagen materials is biodegradation of the implant over time, which necessitates retreatment. Also, similar to the negative reactions to the use of polytetrafluoroethylene particles, collagen has been associated with adverse immune responses and allergic reactions.

Various other injectable substances have been reported or proposed as implant materials for the treatment of bladder conditions, such as vesicoureteral reflux. These substances include polyvinyl alcohol foam, glass particles, a chondrocyte-alginate suspension, and a detachable silicone balloon.

In addition to the various problems associated with many of the substances used to treat urinary incontinence, there are certain disadvantages inherent in the methods currently employed for delivering injectable materials to the periurethral tissue. In particular, the amount of material necessary to compress the urethra must typically be estimated by observing the compression of the urethra wall using a cystoscope or endoscope. If an insufficient amount of material is injected in the first procedure, top-up injections administered in subsequent procedures may be necessary. In addition, the materials which are delivered may be absorbed by the body over time requiring retreatment. Other materials which are used are hydrateable and swell within the body causing difficulty in predicting a final size of the injected material.

U.S. Pat. No. 5,709,854 to Griffith-Cima, et al., U.S. Pat. No. 5,712,252 to Smith, U.S. Pat. No. 5,755,658 to Wallace et al., U.S. Pat. No. 5,763,309 to Milbocker, U.S. Pat. No. 5,785,642 Wallace et al., U.S. Pat. No. 5,855,615 to Bley et al, U.S. Pat. No. 5,976,526 to Atala, U.S. Pat. No. 5,992,025 to Hubbar, U.S. Pat. No. 6,296,607 to Milbocker, and U.S. Pat. No. 6,702,731 to Milbocker, which are incorporated herein by reference in their entirety, describe exemplary bulking compositions and/or methods of injecting the bulking compositions within a mammalian body. In one example, as described in U.S. Pat. Nos. 5,755,658 and 5,785,642, the bulking composition comprises a biocompatible polymer and a biocompatible solvent. The bulking composition is inserted into the periurethral tissue. The biocompatible solvent is miscible or soluble in the fluid of the periurethral tissue and diffuses away upon contact with this fluid. Upon diffusion of the solvent, the biocompatible polymer precipitates to form an occlusion in the periurethral tissue. This formed occlusion compresses the urethra to prevent or reduce the involuntary leakage of urine from the bladder.

In view of the above, it is evident that there is a need for treatment methods, devices, and systems for the treatment of vesicoureteral reflux in mammals which would allow an implant (occlusion)-forming substance to be accurately injected into the subureteral region and which implant-forming substance would preferably be delivered endoscopically, substantially conserve its volume in vivo, be non-migratory and be substantially non-immunogenic.

Further, it would be desirable to provide improved devices, methods, systems, and kits for improving the positioning of bulking compositions adjacent the target periuerthral tissue so that effective occlusions can be accurately formed to avoid the necessity for multiple retreatments.

### SUMMARY OF THE INVENTION

The present invention is directed to the use of implantable, biocompatible compositions or materials to provide bulking at desired locations in a patient's body. The invention is further directed to methods and systems for positioning and delivering such implantable materials.

In one aspect, the materials used, and the methods and systems by which they are delivered and positioned, provide a biocompatible implant, which the body will not reject, that directly acts to control vesicoureteral reflux. The biocompatible implant provides permanent relocation of tissues in the soft tissue area surrounding the desired implant location. The use of the described positioning and delivery system of the present invention provides ease in accurately positioning the implant, which can, for example, result in better placement of the implant for treatment of vesocoureteral reflux. The present invention provides the ease of use of conventional bulking agents, such as, for example, Deflux™, but provides persistent and more predictable bulking than prior art systems and methods for positioning bulking agents.

The methods and systems for positioning and delivering such implantable materials of the present invention can be used in any luminal structure of the body. In one example, the system of the present invention provides a biocompatible implant for treatment of female urinary incontinence. In other aspects, the present invention may be placed into the vascular system to introduce therapeutic drugs to treat cholesterol plaques within the arterial wall; into the digestive tract or biliary tracts to deliver therapeutic drugs or treat diseases; into the lower esophagus to treat gastroesophageal reflux; into the urinary tract to deliver slow release therapeutic drugs. The introduction of slow release therapeutic drugs may apply, for example, to any luminal structure where the drug may act locally or be absorbed preferentially by the organ system and work systemically. The system of the present invention may also be used to guide instruments or devices other than needles as may be needed in various applications (e.g. delivery of a small laser fiber for laser surgery).

### DETAILED DESCRIPTION OF THE FIGURES

The figures are not necessarily to scale, emphasis instead being placed upon illustrating the principles of the invention. These and other features of the preferred embodiments of the invention will become more apparent in the following detailed description in which reference is made to the appended figures wherein:

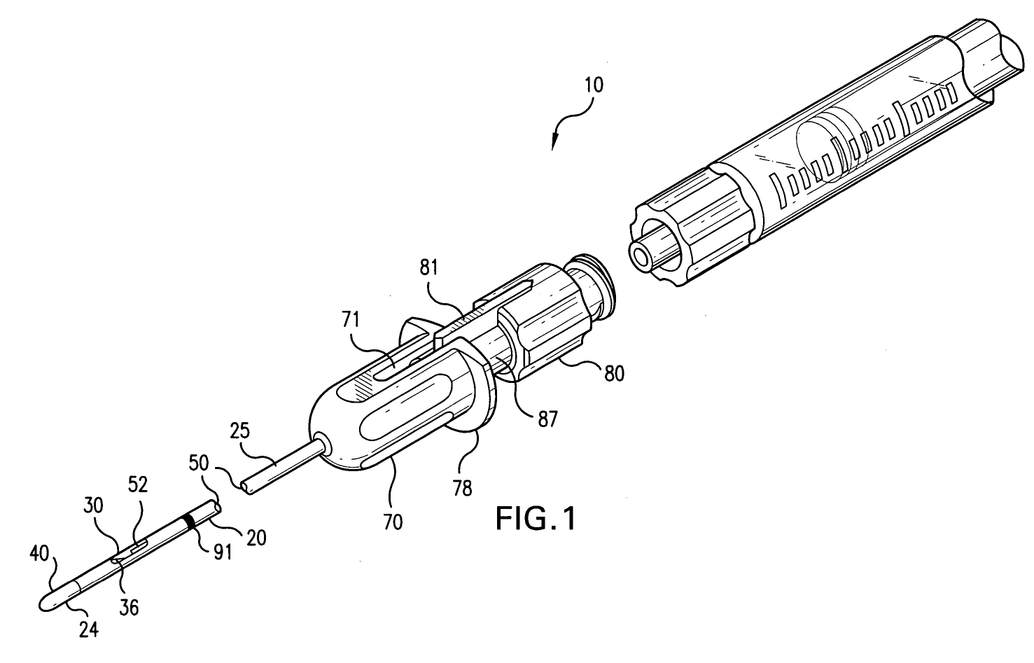

FIG. 1 is a partial perspective view of the implant positioning system of the present invention.

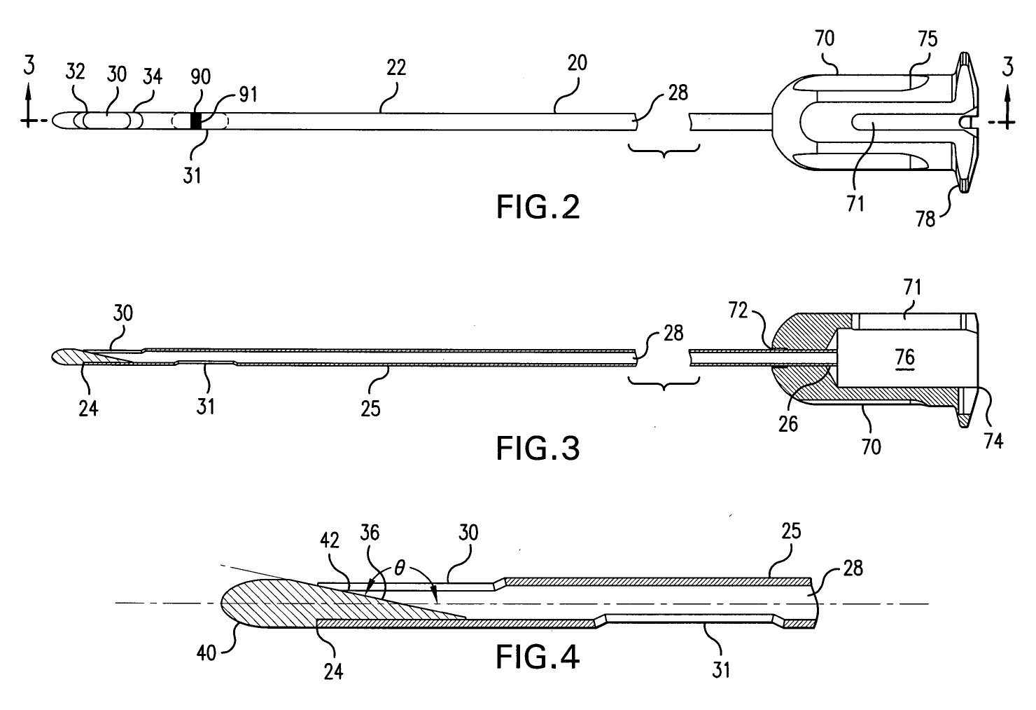

FIG. 2 is a partial top elevational view of one aspect of the cannula of the implant positioning system of FIG. 1, showing a proximal end of the cannula connected to a front end of a cannula hub.

FIG. 3 is a partial cross-sectional view of the cannula and cannula hub of FIG. 2 taken across line 3-3 of FIG. 2.

FIG. 4 is an enlarged cross-sectional view of the tip of the cannula of FIG. 3, showing a first aperture defined in a side wall of the cannula and an angled ramp platform disposed within a cannula lumen.

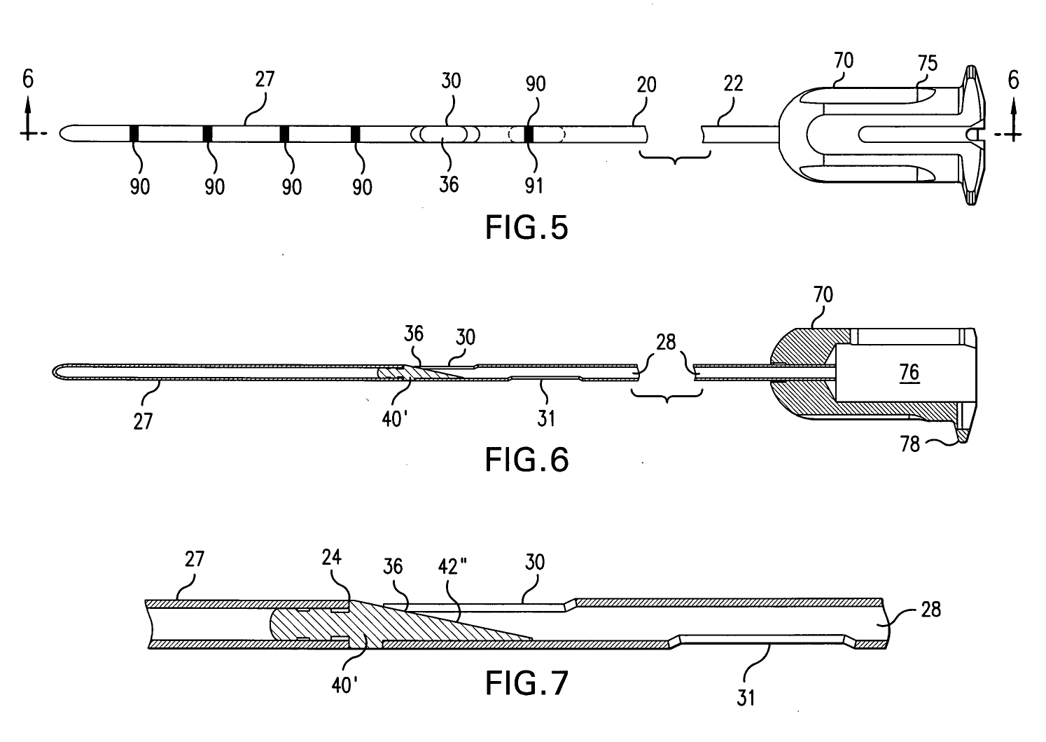

FIG. 5 is a partial top elevational view of one aspect of the cannula of the implant positioning system, showing a proximal end of the cannula connected to a front end of a cannula hub, and further showing a compliant tip portion extending from proximate a first aperture defined in a side wall of the cannula.

FIG. 6 is a partial cross-sectional view of the cannula and cannula hub of FIG. 5 taken across line 6-6 of FIG. 5.

FIG. 7 is an enlarged cross-sectional view of a portion of the cannula of FIG. 6, showing the first aperture defined in the cannula and an angled ramp platform disposed within a cannula lumen.

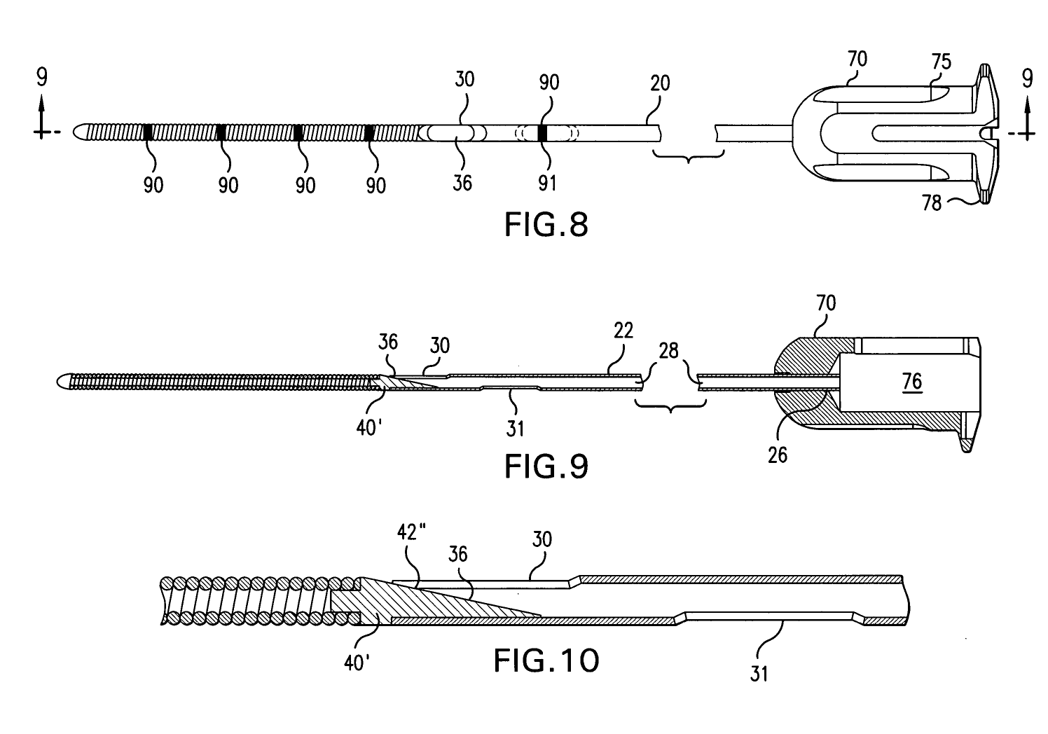

FIG. 8 is a partial top elevational view of one aspect of the cannula of the implant positioning system, showing a proximal end of the cannula connected to a front end of a cannula hub, and further showing a compliant tip portion extending from proximate a first aperture defined in a side wall of the cannula.

FIG. 9 is a partial cross-sectional view of the cannula and cannula hub of FIG. 8 taken across line 9-9 of FIG. 8.

FIG. 10 is an enlarged cross-sectional view of a portion of the cannula of FIG. 9, showing the first aperture of the cannula and an angled ramp platform disposed within a cannula lumen.

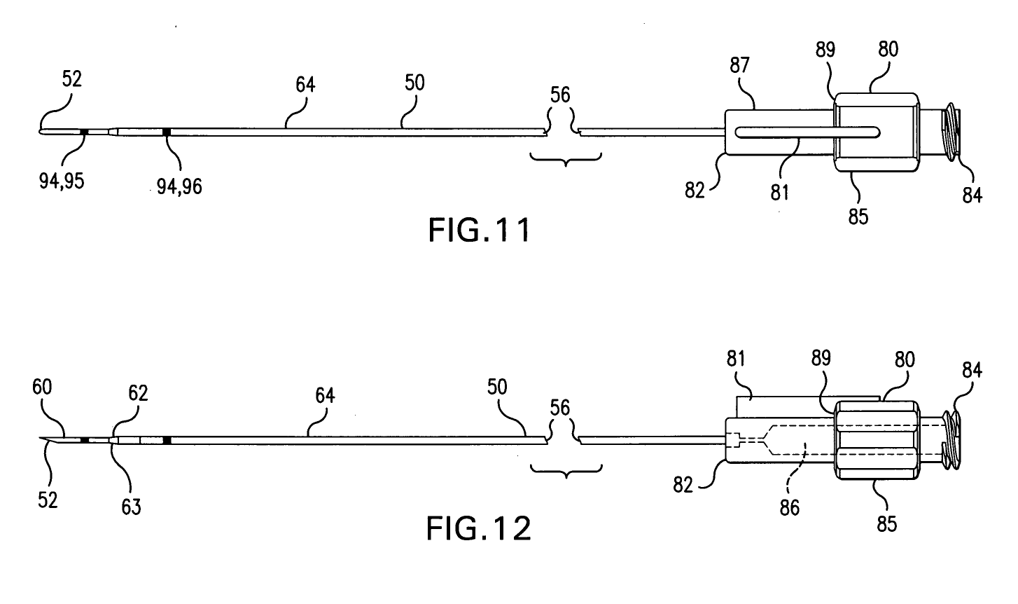

FIG. 11 is a partial top elevational view of the needle of the implant positioning system of FIG. 1, showing an open end of the needle connected to a first end of a needle hub.

FIG. 12 is a partial cross-sectional view of the needle and needle hub of FIG. 11.

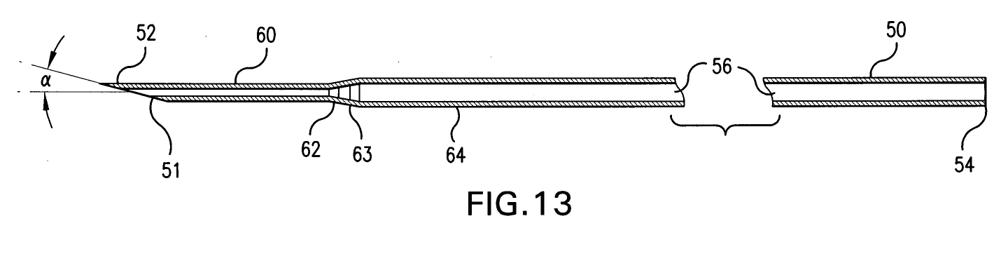

FIG. 13 is a partial cross-sectional view of one aspect of the needle of the present invention.

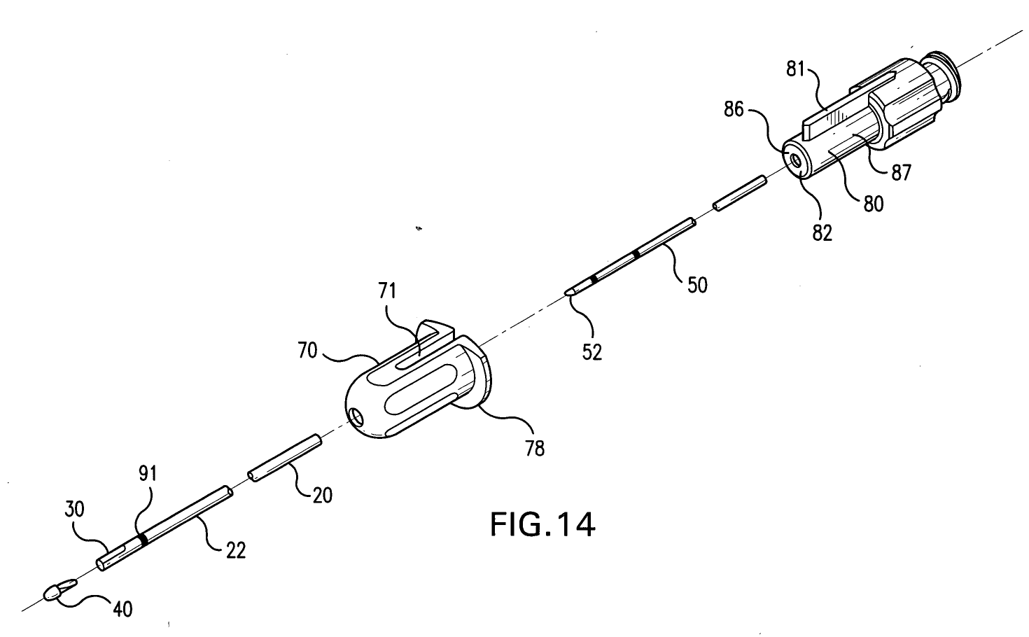

FIG. 14 is an exploded view of alternative aspects of the cannula, the cannula hub, the needle, and the needle hub of the implant positioning system of the present invention.

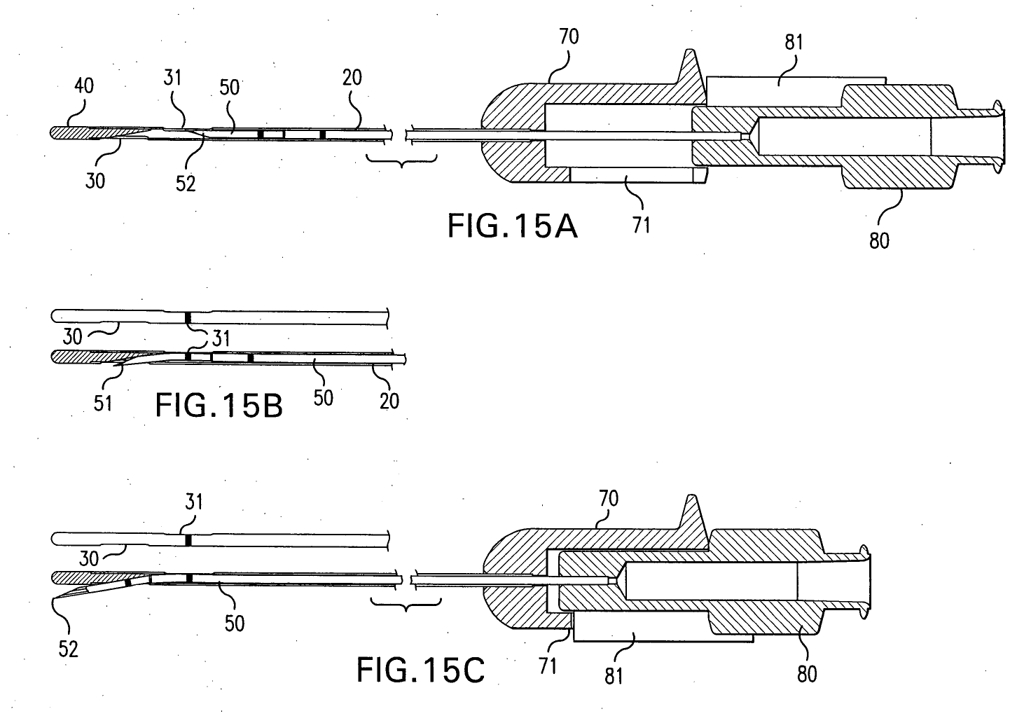

FIGS. 15A-C are partial cutaway side elevational views of one aspect of the implant positioning system of FIG. 1, showing the needle positioned in a withdrawn position, in an initial penetration position, and an extended position.

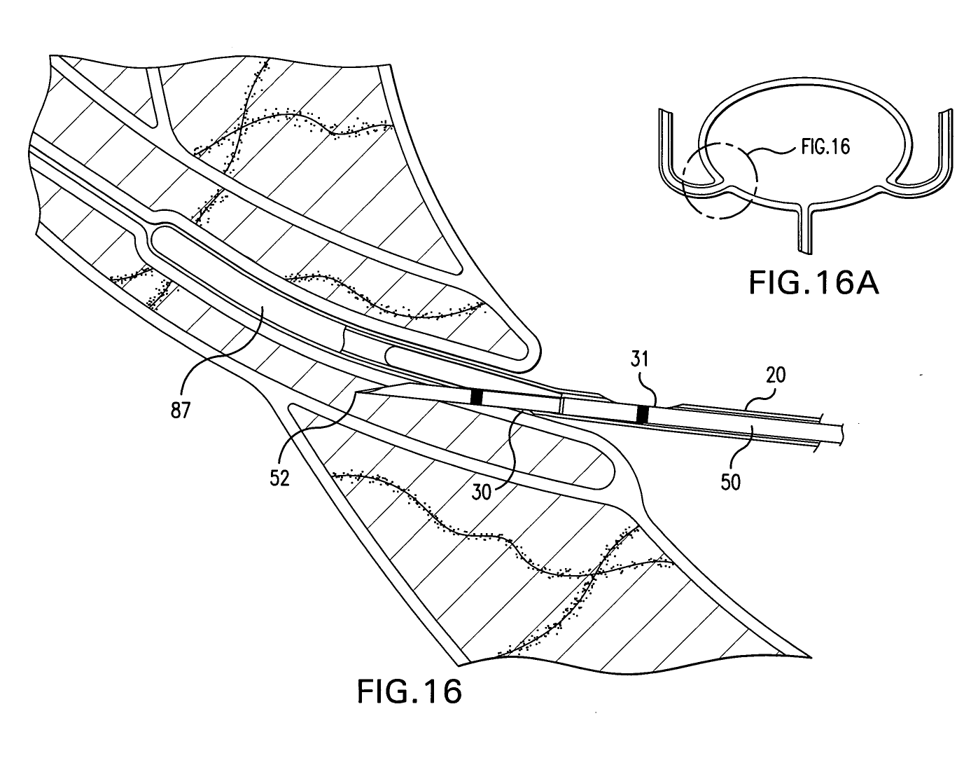

FIG. 16 is a partial cutaway elevational view of an aspect of the implant positioning system of the invention, showing a cannula of a medical device positioned within the ureter and showing a needle in an extended position for delivery of a biocompatible bulking composition.

### DETAILED DESCRIPTION OF THE INVENTION

The present invention may be understood more readily by reference to the following detailed description and any examples provided herein. It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular forms “a,” “an,” and “the” comprise plural referents unless the context clearly dictates otherwise. For example, reference to a component in the singular is intended to comprise a plurality of components.

Ranges may be expressed herein as from “about” or “approximately” one particular value and/or to “about” or “approximately” another particular value. When such a range is expressed, another embodiment comprises from the one particular value and/or to the other particular value. Similarly, when values are expressed as approximations, by use of the antecedent “about,” it will be understood that the particular value forms another embodiment.

As used herein, the term “therapeutic drug” or any other similar term means any chemical or biological material or compound suitable for administration by the methods previously known in the art and/or by the methods taught in the present invention, that induces a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anesthetic effect, or it may be systemic. Such substances include broad classes of compounds normally delivered into the body, including through body surfaces and membranes, including skin. In general, this includes but is not limited to: antiinfectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present invention, both ionized and nonionized drugs may be delivered, as can drugs of either high or low molecular weight.

Referring to the Figures, there is indicated generally by numeral 10 a first aspect of a medical device for guiding the delivery of an injectable material to a predetermined site within a mammal. In one aspect, the injectable material is selected from a group consisting of a biocompatible, non-biodegradable, bulking composition, a biocompatible bulking composition that is at least partially biodegradable, or a therapeutic drug. The medical device 10 comprises a cannula 20 and a needle 50 that is movable therein the cannula. The cannula comprises a generally tubular member 22 having a distal end 24, an open proximal end 26 and a central cannula lumen 28 extending therebetween. The tubular member 22 includes a first aperture 30 through the side wall 25 thereof so as to be in fluid communication with the cannula lumen. The first aperture 30 is positioned a predetermined distance from the distal end 24 of the cannula. Referring to FIGS. 1-4 and 14, in one aspect and with no limitation intended, the predetermined distance between the distal end of the tubular member and the center of the first aperture 30 may be from between about ⅛ in. to about ¾ in, alternatively from between about ⅙ in. to about ½ in, alternatively from between about ¼ in. to about ⅜ in, and alternatively greater than about ¼ in. Referring to FIGS. 5-10 and 14, in other aspects and with no limitation intended, the predetermined distance between the distal end 24 of the tubular member and the center 30 of the first aperture may be from between about ⅙ in. to about 2.0 in., alternatively from between about ¼ in. to about 1.75 in., alternatively from between ¼ in. to about 1.5 in., and alternatively greater than about ¼ in. In one aspect, the first aperture 30 is elongated and has a first aperture end 32 that is positioned closer to the distal end of the cannula than a second aperture end 34. In one example, the first aperture extends generally longitudinally with respect to the longitudinal axis of the tube member.

The cannula 20 further comprises an angled ramp platform 36 that is disposed within the cannula lumen proximate to the first aperture 30. In one example, the angled ramp platform extends proximally and inwardly away from proximate the first aperture end 32 of the first aperture 30. The angled ramp platform forms a predetermined obtuse angle θ relative to the longitudinal axis of the cannula. In one aspect and with no limitation intended, the obtuse angle θ can be between about 150° to about 179°. In one aspect, at least a portion of the ramp platform 36 underlies at least a portion of the first aperture 30. In one aspect, as shown in the figures, a plug 40 is inserted into the end of the tubular member 22 to form the closed distal end 24 of the cannula. In this aspect, a beveled end portion 42 of the plug forms the angled ramp platform 36. Alternatively, in another aspect shown in FIGS. 5-10, a plug 40′ having a beveled end portion is inserted and mounted therein the cannula lumen so that the beveled end portion 42″ is positioned to form the angled ramp platform 36.

In one aspect, the cannula can be formed from a substantially rigid material such as, for example, 300 series stainless steel. In another aspect, the cannula can be formed from a substantially flexible material such as, for example, a polymeric material. In one aspect, at least a portion of the cannula is formed from a transparent material such that at least a portion of the needle disposed therein the cannula lumen is visible. In yet another aspect, shown in FIGS. 5-10 and 14, the cannula can have portions that are flexible and portions that are substantially rigid. In one example, the cannula 20 has a distal portion 27 that extends from about the distal end 24 to about the first aperture 30 that is formed from the flexible material. In this example, the remaining portion of the cannula is formed from the substantially rigid material. In one aspect, the flexible portion of the cannula is mounted onto a distal portion of the plug 40′. The flexible material can be, for example and not meant to be limiting, PVC, wound 300 series stainless steel wire, and the like.

In one aspect, the distal end 24 of the cannula is substantially blunt and can have a smoothly rounded shape. In another aspect, the distal end 24 of the cannula is closed. In an alternative aspect, the distal end of the cannula defines a port [not shown] in communication with the cannula lumen. This port provides for gas sterilization out-gassing or may result from an intentional or inadvertent manufacturing process.

Referring to FIGS. 11-15C, the needle 50 is positioned within the cannula lumen 28 and is movable from and between a withdrawn position and an extended position. The needle has a sharpened open tip 52 to facilitate its penetration through tissue, a spaced open end 54 and a central needle lumen 56 extending therebetween. In one aspect, the open end is in communication with a supply of the injectable material. In its withdrawn position, the sharpened open tip 52 of the needle is stored within the cannula lumen 28. In its extended positioned, the open tip 52 of the needle is directed by the angled ramp platform 36 to extend through the first aperture 30 of the cannula a predetermined distance from the side wall 25 of the cannula. In one aspect and with no limitation intended, the predetermined distance the open tip of the needle extends from the side wall may be from between about 1 mm to about 6 mm, alternatively from between about 1.5 mm to about 5 mm, alternatively between about 2 mm to about 4 mm, and alternatively between about 2.5 mm and 3.5 mm.

It is contemplated that the sharpened open tip 52 can have a tapered cross-sectional shape, a beveled cross-sectional shape, or any other like sharpened cross-sectional shape. In the beveled cross-sectional shape example, the tip can be beveled at a tip angle γ with respect to the longitudinal axis of the needle. In one aspect and with no limitation intended, the tip angle γ may be from between about 5° to about 35°, alternatively, from between about 10° to about 20°, or, alternatively, from between about 14° to about 18°.

In one aspect, the needle 50 has a tip portion 60 that extends a predetermined distance from the sharpened open tip 52 that is connected to a tapered conically-shaped shoulder surface 62 that flares outwardly to form a step 63 in the needle. In one aspect and with no limitation intended, the predetermined distance may be from about 0.35 in. to about 0.45 in. The conically-shaped shoulder is connected to a shaft portion 64 of the needle, which extends from the conically-shaped shoulder to the open end 54 of the needle. The outside diameter of the tip portion 60 is less than the outside diameter of the shaft portion 64 of the needle. Thus, in this aspect, the needle can readily bend about the portions of the needle adjacent to the conically-shaped shoulder surface as the needle is moved between the withdrawn and extended positions.

One will appreciate that the present invention contemplates that the angled ramp platform 36 serves to deflect the needle tip 52 through the first aperture 30 when the needle is moved to the extended position if the cannula is formed from a stiffer material than the needle. Alternatively, the present invention contemplates, if the cannula is formed from a material that is more flexible that the needle, that the needle tip acts against the angled ramp platform to bend the cannula such that the needle tip 52 can exit the first aperture 30 the predetermined distance from the side wall 25 of the cannula. One will also appreciate that the tip portion 60 of the needle extends outwardly and away from the side wall 25 of the cannula 20 at an oblique angle with respect to the longitudinal axis of the cannula.

In one aspect and with no limitation intended, the outside diameter of the tip portion may be from about 0.025 in. to about 0.031 in. and the inside diameter of the tip portion may be from about 0.012 in. to about 0.015 in. Further, in another aspect, the inside diameter of the tip portion can be less than the inside diameter of the shaft portion. In one aspect and with no limitation intended, the outside diameter of the shaft portion may be from about 0.030 in. to about 0.045 in. and the inside diameter of the shaft portion may be from about 0.014 in. to about 0.022 in. In one aspect, the outside diameter of the cannula is sized and shaped to complementarily fit and/or pass through the lumen of a conventional cystoscope (or other endoscopic instrument).

The cannula 20 of the medical device can further comprise a cannula hub 70. In this aspect, the proximal end 26 of the cannula is connected or bonded, such as by soldering, welding, adhering, or other suitable means, to a front end 72 of the cannula hub. The cannula hub 70 defines a hub interior cavity 76 that extends longitudinally from a back end 74 thereof and that is in communication with the proximal end 26 of the cannula. In one aspect, the exterior surface 75 of the cannula hub 70 forms a flange member 78. In one example, the flange member 78 extends substantially transverse to the longitudinal axis of the cannula hub. In another aspect, the flange member extends circumferentially about at least a portion of the exterior surface of the cannula hub.

The needle 50 of the medical device can further comprise a needle hub 80. Here, the open end 54 of the needle is connected or bonded, such as by soldering, welding, adhering, or other suitable means, to a first end 82 of the needle hub 80. The needle hub defines a conduit 86 extending therebetween the first end 82 and a second end 84 such that the conduit 86 of the needle hub 80 is in communication with the needle lumen 56. In one aspect, the second end 84 of the needle hub comprises a means for coupling to a vessel containing a supply or source of the injectable material. In another aspect, the second end of the needle hub forms a conventional syringe attachment, such as, for example, a luer attachment. In another aspect, the exterior surface 85 of the needle hub has a first exterior portion 87 proximate and extending from the first end 82 of the needle hub that is sized and shaped for a complementary slidable fit within the hub interior cavity 76 of the cannula hub 70.

In another aspect, the needle hub comprises a shoulder surface 89 adjoining the first exterior portion 87 intermediate the first and second ends of the needle hub. The shoulder surface extends outwardly away from the first exterior portion of the exterior surface of the needle hub. In use, the shoulder surface 89 of the needle hub 80 can contact the flange member 78 of the cannula hub 70 when the needle is positioned in the extended position. Thus, as one will appreciate, the terminal longitudinal movement of the needle relative to the cannula is limited by the mechanical interference between the shoulder surface and the flange member. In another aspect, the needle hub 80 and the cannula hub 70 can each have shoulder surfaces that are complementarily shaped such that the terminal longitudinal advancement of the needle through the cannula lumen is limited.

In one aspect, the cannula hub 70 can further comprise a slot 71 defined in the exterior surface 75 of the cannula hub that is in communication with the hub interior cavity 76. In one aspect, the slot extends substantially longitudinally from the back end 74 of the cannula hub. In this aspect, the needle hub 80 further comprises a male key 81 extending outwardly away from the exterior surface 85 of the needle hub. In one aspect, the male key extends substantially radially away from the exterior surface of the needle hub. Further, the male key can extend substantially longitudinally between the first and second ends of the needle hub. In another example, at least a portion of the male key 81 extends longitudinally from the first end of the needle hub into at least a portion of the first exterior portion 87 of the needle hub. In another aspect, the end of the male key is spaced is spaced a predetermined distance from the second end of the needle hub. Of course, one will appreciate that it is contemplated that the male key may be formed on the cannula hub and the complementary slot formed on the needle hub.

The slot 71 of the cannula hub is sized and shaped for complementary receipt of the male key 81. Further, the radial distance of a top portion of the male key is greater than the radial distance of the hub interior cavity of the cannula hub. Thus, in use, unless the male key 81 is rotated to align with the slot 71 of the cannula hub, the needle hub 80 cannot be advanced relative to the cannula hub 70 and thus the needle cannot be advanced. As one will appreciate, the needle hub 80 can be advanced relative to the cannula hub 70 when the male key in positioned to align with the slot. In another aspect, the end of the male key is spaced from the second end of the needle hub at a distance at which the needle is in its maximum desired extended position and at which the end of the male key of the needle hub contacts the end of the slot of the cannula hub.

In one aspect, the cannula 20 is connected to the cannula hub 70 such that the angled ramp platform 36 of the cannula lies in a plane substantially transverse to a plane bisecting the slot 71 in the cannula hub and the longitudinal axis of the cannula. In another aspect, in which the open tip of the needle has a beveled cross-sectional shape (thus forming an angled face), the needle 50 is mounted to the needle hub 80 so that the angled face of the open tip of the needle lies in a plane substantially transverse to a plane bisecting the male key and the longitudinal axis of the needle. Thus, in use, when the male key of the needle hub and the slot in the cannula hub are positioned so that they are co-planer with respect to each other, the angled face 51 of the open tip 52 of the needle 50 substantially faces the angled ramp platform 36 of the cannula 20. When the needle is advanced longitudinally toward the extended position (with the male key of the needle hub disposed therein the slot of the cannula hub), a portion of the tip portion of the needle is pushed out of the first aperture such that the sharpened distal end edge of the open tip 52 is the first portion of the needle 50 to extend beyond the side wall 25 of the cannula and penetrate the tissue at an oblique angle at the desired anatomical location.

The cannula 20 can also comprise a second aperture 31 in the side wall 25 that is positioned intermediate the first aperture 30 and the proximal end 26 of the cannula. In one aspect, the second aperture is elongated and extends generally longitudinally with respect to the longitudinal axis of the tube member. In another aspect, the second aperture can be positioned on the side wall of the cannula such that it is not in a plane that bisects the first aperture and the longitudinal axis of the cannula. Alternatively, the second aperture can be positioned on the opposite side of the side wall such that it positioned in a plane bisecting the first aperture and the longitudinal axis of the cannula.

The exterior surface of the side wall 25 of the cannula can have at least one cannula marking 90 thereon. In one aspect, a first cannula marking 91 of the at least one cannula marking is positioned proximate a portion of the second aperture 31. Further, the exterior surface of the needle can have at least one needle marking 94 thereon. As noted above, the needle can comprise a tip portion extending from the needle a predetermined distance. In this example, a first needle marking 95 of the at least one needle marking is positioned on the exterior surface of the tip portion of the needle. A second needle marking 96 can be positioned on the exterior surface of the portion of the needle that adjoins the tip portion. In the described aspect, the first needle marking 95 is positioned adjacent the first cannula marking 91 when the needle is in the withdrawn position and the second needle marking 96 is positioned adjacent the first cannula marking 91 when the needle is in the extended position.

One will appreciate that the methods and systems for positioning and delivering such implantable materials of the present invention can be used in any luminal structure of the body. In one example, the system of the present invention provides a biocompatible implant for treatment of female urinary incontinence. In this example, female incontinence has been treated by periurethral injection therapy, in which the medical device of the present invention is positioned so that the injectable material is injected into the tissue surrounding the urethra, i.e., the periurethral tissue, to add bulk to this tissue.

In other aspects, the present invention may be placed into the vascular system to introduce therapeutic drugs to treat cholesterol plaques within the arterial wall; into the digestive tract or biliary tracts to deliver therapeutic drugs or treat diseases; into the lower esophagus to treat gastroesophageal reflux; into the urinary tract to deliver slow release therapeutic drugs. The introduction of slow release therapeutic drugs may apply, for example, to any luminal structure where the drug may act locally or be absorbed preferentially by the organ system and work systemically. The system of the present invention may also be used to guide instruments or devices other than needles as may be needed in various applications (e.g. delivery of a small laser fiber for laser surgery).

In one particular aspect, the implantable materials used, and the methods and systems by which they are delivered and positioned, provide a biocompatible implant, which the body will not reject, that directly acts to control vesicoureteral reflux. The biocompatible implant provides permanent relocation of tissues in the soft tissue area surrounding the desired implant location. The use of the described positioning and delivery system of the present invention provides ease in accurately positioning the implant, which can, for example, result in better placement of the implant for treatment of vesocoureteral reflux. Further, the present invention provides the ease of use of conventional bulking agents, such as, for example, Deflux™, but provides persistent and more predictable bulking than prior art systems and methods for positioning bulking agents. It will be appreciated that any conventional biocompatible bulking agents are contemplated for use with the medical device of the present invention.

Previously published guidelines on the treatment of VUR, including guidelines from the American Urological Association (AUA) published in 1997, did not include endoscopic injection as a standard treatment option. Long-term use of antibiotics to avoid urinary tract infection was recommended, with children suffering from persistent infections referred for open surgery. Endoscopic injection was not considered appropriate for general use due to a lack of evidence of long-term efficacy, a problem associated particularly with the use of collagen and safety concerns such as migration from the site of injection and tissue reactions, associated with the commonly used agents including PTFE and silicone.

The potential of endoscopic injection as an alternative treatment option has been recognised for some years, as it avoids the need for long-term medication and the potential risks of open surgery. However, an improved injectable agent was required to make this treatment suitable for use in patients with VUR. In recent years, a wealth of data have emerged demonstrating long-term safety and efficacy of a commercially available biocompatible composition called Deflux™ gel manufactured by Q-MED of Uppsala, Sweden. Based on these data, it has been suggest in the art that Deflux™ gel be included as a standard treatment option for VUR—indeed, it has been recommended as first-line treatment for most cases.

Deflux™ gel, used as first line treatment for VUR, can cure the majority of children in a single injection. Repeat injections can be given if the initial treatment does not give an adequate effect. Of course, injection of Deflux™ gel does not preclude future surgical procedures to correct VUR if they are required.

Deflux™ gel is a viscous substance consisting of two components: dextranomer (Dx) microspheres and a gel containing 1% non-animal stabilised hyaluronic acid (NASHA). Both components are made up of polysaccharides—sugar-based molecules. The hyaluronic acid undergoes a mild stabilisation process to cross-link the molecules into a gel, using an agent with minimal risk of biological reactions. A maximum of 1% of the hyaluronic acid molecules are cross-linked. This stabilisation process increases the viscosity and stability of the hyaluronic acid, while retaining biocompatibility. Dextranomer microspheres are formed by cross-linking dextran polymers into porous beads 80-250 μm in diameter. The large size of the dextranomer microspheres minimises any risk of the microspheres migrating from the site of injection to other areas of the body. These dextranomer beads are contained within a gel that is made up of cross-linked polymers of NASHA. Deflux™ gel can also described, therefore, as NASHA/Dx gel.

Both constituents of Deflux™ gel are biocompatible. This means that the material does not cause any significant responses or reactions within the body. The physical and chemical characteristics of NASHA are very similar to the hyaluronic acid found naturally within the body, while the dextranomer is cross-linked, leaving no free dextran molecules that could promote an immune response. The biocompatibility of Deflux™ gel also means there is no risk of long-term accumulation within other organs. The physiochemical properties of Deflux™ gel were optimised for ease of use, high efficacy and safety in the treatment of VUR. As noted and as contemplated, alternative injectable compositions are available for use with the medical device of the present invention such as, for example, silicone and polytetrafluoroethylene (PTFE), but Deflux™ gel is the preferred commercially available biologically compatible composition for the treatment of VUR.

Further, it has been found that Deflux™ is not associated with migration, toxic reactions or any other adverse events or safety concerns, which is an advantage over other materials investigated for endoscopic treatment of VUR. In order to achieve long-term correction of VUR, the implant must remain in position and not disappear over time. Following injection of Deflux™ gel, fibroblast cells have been shown to infiltrate the implant and migrate between the dextranomer microspheres. This is followed by generation of a matrix of collagen which surrounds the microspheres. This effectively replaces the hyaluronic acid component of the implant, which degrades within the first 12 weeks of injection. The position and size of the implant is therefore stabilised long-term. Studies measuring the implant volume over time following Deflux™ gel injection into rats have shown a decrease of just 23% over 12 months. Significantly, a volume decrease of 19% has also been observed in patients 3 months following Deflux™ gel injection. Despite the biodegradability of the constituents of Deflux™ gel, the implant volume is stabilised in the long term by infiltration of endogenous materials.

One well know method for endoscopic injection of Deflux™ gel to treat VUR has been practised for many years and was initially developed for injection of PTFE. In this prior art method, the implant is positioned just below the opening to the ureter (the ureteral orifice) so the technique is commonly referred to as the subureteric transurethral injection or “STING” procedure, devised by O'Donnell and Puri. Injection just below the ureter creates a bolus that increases the submucosal length of the ureter and may also act as a fixation point. The procedure is performed with the child under general anaesthesia, and placed in a lithotomy position to allow access to the bladder. The bladder is semi-filled to allow good visualisation via the cystoscope and to provide support to the implant. This prevents spreading and flattening as the Deflux™ gel is injected.

Prior to injection of Deflux™ gel, a saline solution is flushed through the needle to reduce the resistance to the passage of the gel when pressure is applied to the end of the syringe. A needle is then attached to a pre-filled syringe containing Deflux™ gel. In order to remove any air, the gel is pushed into the needle until a droplet forms at the end. Access to the ureteral orifice is gained via a conventional cystoscope, such as a Wolf 7.5 Fr straight working channel cystoscope with an offset lens (50 optic). The needle is inserted into the working channel of the cystoscope and the cystoscope is introduced into the bladder via the urethra.

The bladder is emptied and is refilled until it is semi-full. This facilitates good visualization and will prevent flattening and spreading of the injected material from high intravesical pressure. The bladder is not overfilled to avoid making the ureteral orifices difficult to inject.

Deflux™ gel is injected into the bladder mucosa, 2-3 mm below the entrance of the ureteral orifice at about the 6 o'clock position. The needle is inserted to a depth of 1-2 mm and the bladder mucosa is lifted over the entire area around the orifice. Deflux™ gel is injected into the submucosal plane. Mild pressure is applied on the syringe, until a distinct mound is created. The STING method will produce a crescent-shaped orifice with a volcano-shaped bolus immediately below.

Following injection of Deflux™ gel, the needle can be held in position for about 15 seconds to prevent leakage from the bolus. For this exemplary implant material, the hyaluronic acid component of the implant creates a seal at the injection site, so there is no need to leave the needle in position for an extended period of time. The entire STING procedure can be expected to take 30 minutes. The bladder is then emptied and the cystoscope removed.

The prior art STING procedure is effective and will cure or improve VUR in the majority of children. However, the present invention includes a method of treating VUR that creates a more effective implant and further increase the success rate. One step of the method of the present invention includes the use of hydrodistention, in which a pressured stream of irrigation fluid passed into the ureter. The method of the present invention is therefore referred to as the hydrodistention-implantation method or “HIT.”

The HIT method of the present invention involves a change in the positioning of the implant. Here the needle is placed within the ureteric tunnel rather than below the ureteral orifice and Deflux™ is injected into the submucosal intraureteric space along the entire length of the detrusor tunnel. As a result, a flap valve mechanism, and perhaps to a lesser degree both hydrostatic and nipple valve mechanisms, are created. The implanted material initially raises the floor of the ureter then moves around the ureteric tunnel, to create a circular bolus that gives complete coaptation and therefore excellent overall success in the treatment of VUR.

Even in cases where there is little or no detrusor backing or tunnel, as in higher grades of VUR, the HIT method permits maximal ureteral coaptation, since the submucosal bulking is within the ureteral wall. As a result, it is technically easier to perform the HIT method in ureters with higher grades of VUR, since the needle may be more feasibly placed in an advantageous position within the ureteral submucosal space.

While mainly carried out according to the STING methodology described above, the HIT methodology includes the use of a sufficiently large cystoscope to allow hydrodistention. In one example, a Wolf 9.5 Fr straight working cystoscope with a 50 offset lens is used. A glass rod lens provides optimal visualization. The bladder is filled to 50% to 75% of its volume to permit visualization of the ureter and avoid both distortion and tension within the submucosal layer of the ureter secondary to over-distention.

First, a pressured stream of irrigation fluid is directed into the ureter (hydrodistention) to define the site of injection within the ureteral submucosa within the detrusor tunnel. Ureteral hydrodistension caused the ureteral orifice to open pre-treatment, remain closed post-treatment, and aid in obtaining a successful implantation. The distal end of the medical device of the present invention is inserted into the ureter of the patient and is oriented so that the first aperture of the cannula is positioned adjacent the targeted anatomical site (generally, within the submucosa of the mid to distal ureteral tunnel at about the 6 o'clock position). The needle hub and cannula hub are positioned such that the male key of the needle hub and the slot of the cannula hub are co-planer with respect to each other and the needle is then moved from the withdrawn position to the extended position such that the needle tip is inserted obliquely into the ureteral submucosa to a desired depth, for example and not meant to be limiting, a depth of between about 1 to about 6 mm, between about 3 to about 5 mm, or about 4 mm. The respective positions of the needle and cannula markings relative to each other can be used to determine the relative depth of the needle tip.

The hydrodistention is stopped during Deflux™ gel injection to allow any leakage of the gel into the ureteric tunnel to be visualised. A small volume of Deflux™ gel is injected initially to check the position of the implant. Once the needle is placed, upward rotation of the needle and ureteral orifice is avoided as torque may obscure the visual clues needed to determine proper ureteral tunnel coaptation. The cystoscope is then retracted to the bladder neck to allow the physician to trace the movement of the material around the ureter while a larger volume is injected (for example and not meant to be limiting, between about 0.3 ml to about 2.0 ml in total, or between about 0.5 to about 1.5 ml in total) to achieve complete coaptation. Generally, substantially the entire ureteric tunnel will rise up during the injection of Deflux™. By placing the needle in the submucosal plane, tracking of material cephalad within the ureteral submucosa occurs. This allows for the maximum length of ureteral coaptation. As the needle is pulled distally, further injection can make the ureteral orifice appear substantially completely coapted.

Following treatment for VUR, nearly all patients are maintained on antibiotic prophylaxis until absence of reflux is confirmed. The HIT procedure has increased the overall success rates of Deflux™ gel injection and can also be used to treat complicated VUR cases without significant reductions in efficacy. When the HIT procedure is compared to the STING procedure, a significant improved cure rate (89% vs. 71%) was achieved for the HIT procedure. See Kirsch, A. et al., J. Urology, 2004. Patients with numerous complications, including some that have been excluded from previous analyses such as failed surgery, neurogenic bladder and hutch diverticulum, were investigated. An overall success rate (reflux grade 0) of 68% with one HIT treatment was observed in the 69 patients assessed after 3 months. A variation in the success rate was observed between the complications. Deflux™ gel injection cured reflux (grade 0) in 88% of the 17 patients in whom open surgery had previously failed, and the success rate in patients with double ureters was 73% (15 patients). The treatment was well tolerated in all patient groups, with no cases of post-treatment pyelonephritis or urinary retention.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
a cannula having a distal end, an open proximal end, and defining a cannula lumen extending therebetween, the cannula having a longitudinal axis, the cannula defining a first aperture in a side wall thereof in communication with the cannula lumen, the first aperture being positioned a predetermined distance from the distal end of the cannula and having a first aperture end and an opposed second aperture end, the first aperture end being closer to the distal end of the cannula than the second aperture end, the cannula further comprising an angled ramp platform disposed within the cannula lumen proximate to the first aperture end, the angled ramp platform extending proximally and inwardly away from proximate the first aperture end of the aperture at a predetermined obtuse angle relative to the longitudinal axis of the cannula; and
a needle positioned within the cannula lumen and movable therein, the needle having an open tip, a spaced open end in fluid communication with a biocompatible bulking composition, and defining a needle lumen extending therebetween for delivering the biocompatible bulking composition to a patient, the needle being movable from and between a withdrawn position, at which the tip of the needle is stored within the cannula lumen, to an extended position, at which the tip of the needle extends through the first aperture of the cannula a predetermined distance from the side wall of the cannula.

2. The medical device of claim 1, wherein the cannula further comprises a cannula hub having a front end, an opposed back end, and a longitudinal axis, the proximal end of the cannula being connected to the front end of the cannula hub, the cannula hub defining a hub interior cavity that extends longitudinally from the back end thereof and that is in communication with the proximal end of the cannula.

3. The medical device of claim 2, wherein the needle further comprises a needle hub having a first end, a spaced second end, and a longitudinal axis, the needle hub defining a conduit extending therebetween the first and second ends, the open end of the needle being connected to the first end of the needle hub such that the conduit of the needle hub is in communication with the needle lumen.

4. The medical device of claim 3, wherein the needle hub has an exterior surface, the exterior surface of the needle hub having a first exterior portion proximate the first end that is sized and shaped for a complementary slidable fit within the hub interior cavity of the cannula hub.

5. The medical device of claim 4, wherein the needle hub forms a longitudinally extending shoulder surface positioned intermediate the first and second ends of the needle hub, the shoulder surface extending outwardly away from the exterior surface of the needle hub, and wherein at least a portion of the shoulder surface is in the first exterior portion.

6. The medical device of claim 5, wherein the shoulder surface is spaced a predetermined distance from the first end of the needle hub.

7. The medical device of claim 3, wherein the second end of the needle hub forms a luer attachment.

8. The medical device of claim 3, wherein the second end of the needle hub comprises a means for coupling to a vessel containing a source of the biocompatible bulking composition.

9. The medical device of claim 3, wherein the cannula hub has an exterior surface, and wherein a portion of the exterior surface of the cannula hub forms a flange member that extends outwardly substantially transverse to the longitudinal axis of the cannula hub.

10. The medical device of claim 9, wherein the flange member extends circumferentially about at least a portion of the exterior surface of the cannula hub.

11. The medical device of claim 3, wherein the cannula hub defines a slot in an exterior surface of the cannula hub that is in communication with the hub interior cavity, the slot extending longitudinally from the back end of the cannula hub, and wherein the needle hub defines a male key extending radially away from a portion of an exterior surface of the needle hub, the male key extending substantially longitudinally from the first end of the needle hub, wherein the radial distance of a top portion of the male key is greater than the radial distance of the hub interior cavity of the cannula hub.

12. The medical device of claim 1, wherein the needle comprises a tip portion extending from the tip of the needle a predetermined distance, and wherein the tip portion has an exterior diameter that is less than the exterior diameter of an adjoining portion of the needle such that a step in the needle is formed therebetween.

13. The medical device of claim 1, wherein the first aperture is elongated and extends substantially longitudinally.

14. The medical device of claim 1, wherein the cannula defines a second aperture in the side wall, the second aperture being positioned intermediate the first aperture and the proximal end of the cannula.

15. The medical device of claim 14, wherein the second aperture is elongated and extending substantially longitudinally.

16. The medical device of claim 14, wherein the second aperture is positioned on the opposite side of the side wall relative to the first aperture.

17. The medical device of claim 14, wherein an exterior surface of the side wall of the cannula has at least one cannula marking thereon.

18. The medical device of claim 17, wherein a first cannula marking of the at least one cannula marking is positioned proximate a portion of the second aperture.

19. The medical device of claim 18, wherein an exterior surface of the needle has at least one needle marking thereon.

20. The medical device of claim 19, wherein the needle comprises a tip portion extending from the tip of the needle a predetermined distance, wherein a first needle marking of the at least one needle marking is positioned on the exterior surface of the tip portion of the needle, and wherein a second needle marking of the at least one needle marking is positioned on the exterior surface of the portion of the needle that adjoins the tip portion.

21. The medical device of claim 20, wherein the first needle marking is positioned adjacent the first cannula marking when the needle is in the withdrawn position, and wherein the second needle marking is positioned adjacent the first cannula marking when the needle is in the extended position.

21. The medical device of claim 1, wherein the distal end of the cannula is blunt.

22. The medical device of claim 21, wherein the distal end of the cannula is smoothly rounded.

23. The medical device of claim 21, wherein the distal end of the cannula is closed.

24. The medical device of claim 1, wherein the distal end of the cannula defines a port in communication with the cannula lumen.

25. The medical device of claim 1, wherein at least a portion of the ramp platform underlies at least a portion of the first aperture.

26. The medical device of claim 1, wherein the cannula is formed from a substantially rigid material.

27. The medical device of claim 1, wherein the cannula is formed from a substantially flexible substance.

28. The medical device of claim 1, wherein the cannula has a distal portion extending from between about the distal end of the cannula and the first aperture, wherein at least the distal portion is formed from a substantially flexible material.

29. A medical device, comprising:
a cannula having a distal end, an open proximal end, and defining a cannula lumen extending therebetween, the cannula having a longitudinal axis, the cannula defining a first aperture in a side wall thereof in communication with the cannula lumen, the first aperture being positioned a predetermined distance from the distal end of the cannula and having a first aperture end and an opposed second aperture end, the first aperture end being closer to the distal end of the cannula than the second aperture end, the cannula further comprising an angled ramp platform disposed within the cannula lumen proximate to the first aperture, the angled ramp platform extending proximally and inwardly away from proximate the first aperture end of the aperture at a predetermined obtuse angle relative to the longitudinal axis of the cannula;
a needle longitudinally movable from and between a withdrawn position, at which a tip of the needle is stored within the cannula lumen, to an extended position, at which the tip of the needle extends through the first aperture of the cannula a predetermined distance from the side wall of the cannula.

30. The medical device of claim 29, further comprising a supply of injectable material that is connected to the needle.

31. The medical device of claim 30, wherein the supply of injectable material is selected from a group consisting of: a biocompatible, non-biodegradable, bulking composition; a biocompatible bulking composition that is at least partially biodegradable; or drugs.

32. A system for delivering a biocompatible bulking composition to a urological site, the system comprising:
a cannula defining an aperture in a side wall thereof and comprising an angled ramp platform disposed within the cannula proximate to the aperture, the angled ramp platform extending proximally and inwardly away from proximate the distal end of the aperture at a predetermined obtuse angle relative to a longitudinal axis of the cannula;
a needle longitudinally movable within the cannula between a withdrawn position, at which a tip of the needle is stored within the cannula, to an extended position, at which the tip of the needle extends through the aperture of the cannula a predetermined distance from the side wall of the cannula.

33. The system of claim 32, further comprising a supply of injectable biocompatible bulking composition for controlling vesicoureteral reflux connected to the needle.

34. The system of claim 33, further comprising a syringe that is releasably connected to the needle, wherein the supply of injectable biocompatible composition is stored within the syringe.

35. The system of claim 32, further comprising a cystoscope defining a bore, wherein the cannula is longitudinally movable within the bore of the cystoscope.

36. A method of treating visicoureteral reflux in a mammal, comprising:
a. providing a medical device comprising:
i) a cannula having a distal end, an open proximal end, and defining a cannula lumen extending therebetween, the cannula having a longitudinal axis, the cannula defining a first aperture in a side wall thereof in communication with the cannula lumen, the first aperture being positioned a predetermined distance from the distal end of the cannula and having a first aperture end and an opposed second aperture end, the first aperture end being closer to the distal end of the cannula than the second aperture end, the cannula further comprising an angled ramp platform disposed within the cannula lumen proximate to the first aperture, the angled ramp platform extending proximally and inwardly away from proximate the first aperture end of the aperture at a predetermined obtuse angle relative to the longitudinal axis of the cannula; and
ii) a needle positioned within the cannula lumen and movable therein, the needle having an open tip, a spaced open end in fluid communication with a biocompatible bulking composition, and defining a needle lumen extending therebetween for delivering the biocompatible bulking composition to a patient, the needle being movable from and between a withdrawn position, at which the tip of the needle is stored within the cannula lumen, to an extended position, at which the tip of the needle extends through the first aperture of the cannula a predetermined distance from the side wall of the cannula;
b. directing a pressurized stream of irrigation fluid into the ureter to open the ureteral orifice;
c. inserting the distal end of the cannula of the medical device through the ureteral orifice and into the ureteral tunnel;
d. orienting the first aperture of the cannula so that it is adjacent a targeted anatomical site;
e. moving the needle from the withdrawn position toward an at least partially extended position to insert the needle tip into the targeted anatomical site to a desired depth; and
f. injecting a volume of the biocompatible bulking composition into the targeted anatomical site to achieve coaptation.

37. The method of claim 36, further comprising:
withdrawing the needle by moving the needle from the at least partially extended position to the withdrawn position;
withdrawing the cannula from the ureter; and
withdrawing the medical device from the mammal.

38. The method of claim 36, further comprising stopping the pressurized stream of irrigation fluid prior to the injection of the biocompatible bulking composition.

39. The method of claim 36, further comprising, prior to directing the steam of irrigation fluid into the ureter, filling the bladder of the mammal to between about 50% to about 75% of its volume.

40. The method of claim 36, further comprising:
inserting the medical device into a cystoscope prior to inserting the cannula of the medical device into the ureteral tunnel; and
retracting the cystoscope to proximate the bladder neck prior to the injection of the biocompatible bulking composition.

41. The medical device of claim 36, wherein the targeted anatomical site is within the ureteral submucosa of the mid to distal ureteral tunnel.

42. The medical device of claim 41, wherein the targeted anatomical site is at about the 6 o'clock position in the ureteral tunnel.

43. The medical device of claim 41, wherein the needle tip is inserted obliquely into the ureteral submucosa to the desired depth.

44. The method of claim 36, wherein the desired depth is between about 3 mm to about 5 mm.

45. The method of claim 36, wherein the volume of biocompatible bulking composition injected is between about 0.3 ml to about 2.0 ml.

46. The method of claim 44, wherein the biocompatible bulking composition is Deflux™.
